(19) United States
(12) Patent Application Publication
(10) Pub. No.: US 2026/0215757 A1
(43) Pub. Date: Jul. 30, 2026

(54) WEARABLE ULTRASOUND APPARATUS

(71) Applicant: Dawako Medtech, S.L., Paterna (Valencia) (ES)

(72) Inventors: Agustin Macia Barber, Paterna (Valencia) (ES); Victor Manuel Garcia Chocano, Paterna (Valencia) (ES); Eduardo Jorge Godoy, Paterna (Valencia) (ES)

(73) Assignee: Dawako Medtech, S.L., Paterna (Valencia) (ES)

(21) Appl. No.: 19/570,083

(22) Filed: Mar. 17, 2026

Related U.S. Application Data
(63) Continuation of application No. 17/794,928, filed on Jan. 21, 2021.
(60) Provisional application No. 62/965,276, filed on Jan. 24, 2020.

(51) Int. Cl. A61B 8/00; A61B 5/00; A61B 5/367

(52) U.S. Cl. CPC A61B 8/4416; A61B 5/0075; A61B 5/367; A61B 8/4236; A61B 8/4281; A61B 8/4477; A61B 8/4488

(57) ABSTRACT

A wearable ultrasound apparatus is disclosed for use in connection with various biomedical applications, including musculoskeletal (“MSK”) imaging and analysis. In at least one embodiment, the apparatus provides at least one of an ultrasound module configured for obtaining an at least one ultrasound image of a portion of a user's body on which the at least one ultrasound module is positioned (hereinafter referred to as the “target site” for simplicity purposes), a electrophysiological (“EP”) module configured for detecting bioelectric signals of the target site, and a near-infrared spectroscopy (“NIRS”) module configured for monitoring oxygenation status and/or biochemical measurements of the target site.

### RELATED APPLICATIONS

This application is a Continuation of U.S. patent application Ser. No. 17/794,928 filed Jul. 22, 2022 which claims priority and is entitled to the filing date of U.S. provisional application No. 62/965,276, filed on Jan. 24, 2020. The contents of the aforementioned applications are incorporated herein by reference.

### BACKGROUND

The subject of this patent application relates generally to ultrasound devices, and more particularly to a wearable ultrasound apparatus configured for use in connection with various biomedical applications.

Applicant(s) hereby incorporate herein by reference any and all patents and published patent applications cited or referred to in this application.

By way of background, ultrasound waves are utilized in many different fields, typically as a tool to penetrate a medium to measure its reflection signature. In medicine, ultrasound imaging devices are usually used for diagnostic medical imaging of internal organs, muscles, tendons and other objects positioned within a patient, among other applications. Traditional ultrasound imaging devices are capable of providing sophisticated live images and enable extraction of characteristic features using advanced signal processing techniques. However, they are generally large, stationary and expensive. Moderate size imaging devices with limited mobility, such as computer-on-wheels systems, are also available with performance generally similar to the larger systems. Handheld versions, along with wearable versions, of such devices have also been developed in recent years, which provide relatively more mobility. However, to Applicant's knowledge, none of these known devices are capable of simultaneously acquiring ultrasonic imaging, electrophysiological, hemodynamic, and metabolic information of internal organs, muscles, tendons and other soft tissues of the patient in biomedical and clinical applications.

Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a wearable ultrasound apparatus configured for use in connection with various biomedical applications, including musculoskeletal (“MSK”) imaging and analysis. In at least one embodiment, the apparatus provides at least one of an ultrasound module configured for obtaining an at least one ultrasound image of a portion of a user's body on which the at least one ultrasound module is positioned (hereinafter referred to as the “target site” for simplicity purposes), a electrophysiological (“EP”) module configured for detecting bioelectric signals of the target site, and a near-infrared spectroscopy (“NIRS”) module configured for monitoring oxygenation status and/or biochemical measurements of the target site.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a diagrammatic cross-sectional view of an exemplary wearable ultrasound apparatus, in accordance with at least one embodiment;

FIGS. 2A, 2B and 2C are diagrammatic views of exemplary piezoelectric sensors and electrodes of an exemplary ultrasound transducer, in accordance with at least one embodiment;

FIG. 3 is a further diagrammatic view of exemplary piezoelectric sensors, in accordance with at least one embodiment;

FIG. 4 is a further diagrammatic view of exemplary array of piezoelectric sensors, in accordance with at least one embodiment;

FIG. 4A is a detailed view of the section defined by line 4A of FIG. 4;

FIGS. 5A and 5B are diagrammatic views of exemplary arrays of piezoelectric sensors, in accordance with at least one embodiment;

FIG. 6 is a schematic view of an exemplary wearable ultrasound apparatus, in accordance with at least one embodiment;

FIG. 7 is a schematic view of an exemplary ultrasound transceiver, in accordance with at least one embodiment;

FIG. 8 is a schematic view of an exemplary electrophysiological (“EP”) module, in accordance with at least one embodiment;

FIG. 9 is a schematic view of an exemplary near-infrared spectroscopy (“NIRS”) module, in accordance with at least one embodiment;

FIG. 10 is a diagrammatic cross-sectional view of a further exemplary wearable ultrasound apparatus, in accordance with at least one embodiment; and

FIG. 11 is a diagrammatic cross-sectional view of a still further exemplary wearable ultrasound apparatus, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

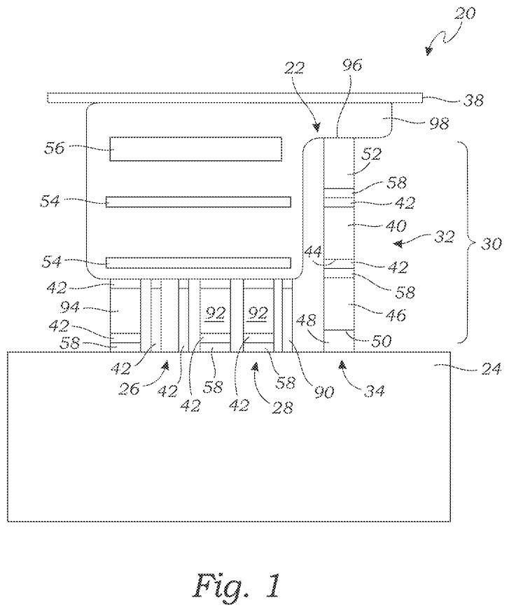

Turning now to FIG. 1, there is shown a diagrammatic cross-sectional view of an exemplary embodiment of a wearable ultrasound apparatus 20 configured for use in connection with various biomedical applications, including musculoskeletal (“MSK”) imaging and analysis. In at least one embodiment, the apparatus 20 provides at least one of an ultrasound module 22 configured for obtaining an at least one ultrasound image of a portion of a user's body on which the at least one ultrasound module 22 is positioned (hereinafter referred to as the “target site” 24 for simplicity purposes), a electrophysiological (“EP”) module 26 configured for detecting bioelectric signals of the target site 24, and a near-infrared spectroscopy (“NIRS”) module 28 configured for monitoring oxygenation status and/or biochemical measurements of the target site 24, each of which are discussed further below. At the outset, it should be noted that the particular arrangement of components illustrated in FIG. 1 is merely exemplary. Thus, in further embodiments, as discussed further below, the various components may take on a number of other arrangements.

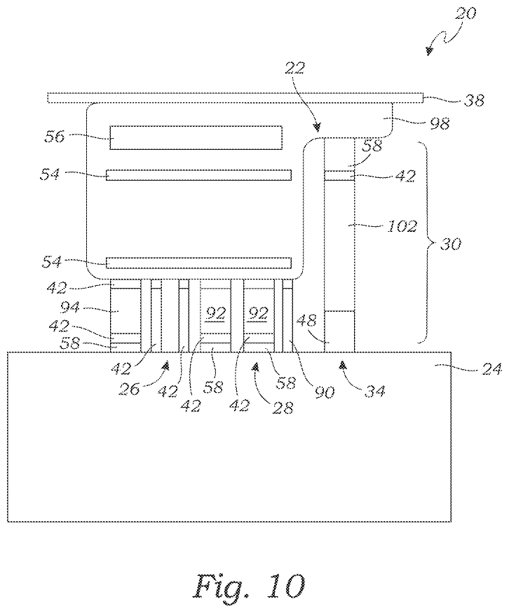

In at least one embodiment, the ultrasound module 22 provides an at least one ultrasound transducer 30 intended to operate in the range of 7-14 MHz for superficial scans and 2-6 MHz for deeper targets. However, in further embodiments, the at least one ultrasound transducer 30 may operate in any other range, now known or later developed, capable of allowing the apparatus 20 to substantially carry out the functionality described herein. Additionally, in at least one embodiment, the at least one ultrasound transducer 30 is configured for operating in a pulse-echo configuration—i.e., it is configured to emit and subsequently receive ultrasonic pulses in order to obtain the at least one ultrasound image. In at least one embodiment, the at least one ultrasound transducer 30 comprises an at least one piezoelectric sensor 32. In at least one such embodiment, the ultrasound transducer 30 comprises a plurality of piezoelectric sensors 32 configured as an at least one array 34, with the quantity of piezoelectric sensors 32 in a given array 34 ranging between 2 and 256. However, in further embodiments, any other quantities of piezoelectric sensors 32 may be utilized. In at least one alternate embodiment, as illustrated in FIG. 10, the at least one ultrasound transducer 30 comprises an at least one microelectromechanical (“MEM”) sensor 102—such as an at least one capacitive micromachined ultrasonic transducer (“CMUT”) or piezoelectric micromachined ultrasonic transducer (“PMUT”), for example—either in addition to or in lieu of the at least one piezoelectric sensor 32. Both CMUT and PMUT are based on the oscillation of a membrane suspended on a cavity developed in a silicon substrate.

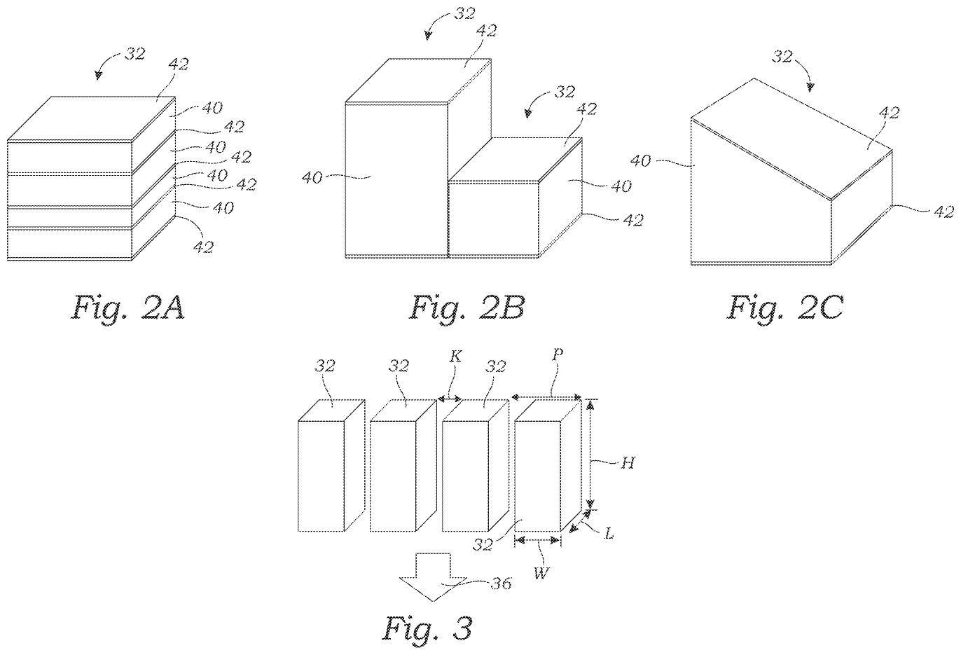

In at least one embodiment, the at least one ultrasound transducer 30 is positioned on an at least one resilient substrate 38. In at least one such embodiment, the resilient substrate 38 is comprised of a material that is flexible and/or stretchable. For example, in at least one such embodiment, the material is comprised of at least one of a silicon-based material, rubber, thermoplastic elastomers, polymeric materials, foils (such as those mixed with epoxy), and various fabrics. In at least one further embodiment, the resilient substrate 38 is comprised of a material that is transparent, flexible, and conformable in nature. Additionally, in at least one embodiment, the material is biocompatible, latex-free, non-toxic, and non-allergenic. In still further embodiments, the resilient substrate 38 may comprise any other materials (or combinations of materials) having flexible and/or rigid-flexible characteristics, now known or later developed, so long as the apparatus 20 is capable of substantially carrying out the functionality described herein. In at least one embodiment, the resilient substrate 38 is made of composite epoxy material (“CEM”), fiberglass, or paper base class materials providing a solid foundation for printed circuit boards (“PCBs”). For example, in at least one such embodiment, the material is comprised of at least one of an epoxy resin (FR4, FR5, FE-3), PF resin (XPC, FR1, FR2), and polyester resin. In at least one alternate embodiment, the resilient substrate 38 is positioned and employed to perform the function of any of the layers of the ultrasound module 22, EP module 26 or NIRS module 28, provided that it is placed at the position of the layer whose function performs and its material properties are suitable. In at least one embodiment, the resilient substrate 38 has a thickness of approximately 180 micrometers or less, such that the apparatus 20 has an overall thickness of approximately 25 millimeters or less. However, in further embodiments, the resilient substrate 38 may have any other thickness, so long as the apparatus 20 is capable of substantially carrying out the functionality described herein. In at least one embodiment, the resilient substrate 38 is configured as a flexible film with signal traces embedded within or over the resilient substrate 38, and the at least one piezoelectric sensor 32 is attached to the resilient substrate 38. In at least one alternate embodiment, the at least one piezoelectric sensor 32 and the signal traces are simultaneously screen-printed onto the resilient substrate 38, which can provide a number of benefits. For example, in at least one such embodiment, simultaneously screen-printing the at least one piezoelectric sensor 32 and the signal traces onto the resilient substrate 38 can reduce the number of steps involved in the manufacturing process. Additionally, the size, shape and arrangement of the at least one piezoelectric sensor 32 (relative to the resilient substrate 38) can be freely customized, depending on the intended use of the apparatus 20 in a given embodiment. Specifically, the shape of the at least one piezoelectric sensor 32 can be modified to have rounded corners, so as to introduce aperture apodization, thereby improving sidelobe suppression. Further exemplary shapes could include (but are in no way limited to) rings, hexagons, circles, rectangles, etc. Additionally, in at least one such embodiment, where the at least one piezoelectric sensor 32 comprises a piezoelectric material 40 sandwiched between two or more electrodes 42 (as illustrated in FIGS. 2A, 2B and 2C, and discussed further below), given that a distance between electrodes 42 (based at least partially on the thickness of the piezoelectric material 40) defines an excitation frequency therebetween, a plurality of electrodes 42 can be implemented with varying distances therebetween so as to enable the at least one ultrasound transducer 30 to operate at multiple frequencies and with improved bandwidth.

In at least one embodiment, the distance between centers of two contiguous piezoelectric sensors 32 of an array 34 is less than approximately 0.5λ for phased array operation and approximately 0.75λ-3λ for linear array operation, where λ=c/f, with λ being the wavelength of the ultrasound signal with frequency f and longitudinal sound speed c≈1500 m/s. Some numeric examples of the aforementioned limits are given in Table 1 below.

TABLE 1

| | | |
|---|---|---|
| 7 MHz | 10 MHz | 14 MHz |

| | | | | | | |
|---|---|---|---|---|---|---|
| 0.5 λ | 107.1 | μm | 75 | μm | 53.6 | μm |
| 0.75 λ | 160.7 | μm | 112.5 | μm | 80.4 | μm |
| 3 λ | 642.9 | μm | 450 | μm | 321.4 | μm |

In at least one embodiment, as illustrated in FIG. 3, each of the piezoelectric sensors 32 has a width W that is relatively less than a pitch P, given that a small kerf K (i.e., a separation) is required between the piezoelectric sensors 32 so as to provide acoustic inter-element isolation to each of the piezoelectric sensors 32. Additionally, in at least one embodiment, each of the piezoelectric sensors 32 has a thickness or height H that is at least partially dependent on the resonant frequency at which the ultrasound transducer 30 operates. Several examples of thickness are shown in Table 2 below for different materials. Additionally, in at least one embodiment, each of the piezoelectric sensors 32 has a length L which is less restricted by design constraints in comparison with the other two dimensions.

TABLE 2

| | | |
|---|---|---|
| 7 MHz | 10 MHz | 14 MHz |

| | | | | | | |
|---|---|---|---|---|---|---|
| PZT-5A | 230.5 | μm | 161.3 | μm | 115.3 | μm |
| PZT-5H | 271.4 | μm | 190 | μm | 135.7 | μm |
| PVDF | 157.1 | μm | 110 | μm | 78.6 | μm |

In at least one embodiment, the thickness/height H of a given piezoelectric sensor 32 is a function of frequency of the sound waves. In at least one such embodiment, each of the piezoelectric sensors 32 has a height H of approximately 300 micrometers or less. However, in further embodiments, each of the piezoelectric sensors 32 may have any other height H, so long as the apparatus 20 is capable of substantially carrying out the functionality described herein.

In at least one embodiment, the at least one piezoelectric sensor 32 may be made of any suitable material, including but not limited to a flexible piezoelectric coating (film, paste or paint), a ceramic transducer, or a polymer block transducer. Additionally, in at least one embodiment, the at least one piezoelectric sensor 32 may be comprised of quartz, polyvinylidene fluoride, ceramic including PZT and screen-printed ceramic, magneto strictive, or composite material including molded ceramic and benders. For example, the piezoelectric material may be selected from the group consisting of Polyvinylidene fluoride (PVDF) and its co-polymers, lead zirconate titanate Pb(Zr,Ti)O3, lead metaniobate Pb(Nb2O6), modified lead titanate PbTi3, (Pb, Ca)Ti03, (Pb, Sm)Ti03, barium titanate BaTiO3, PMN-PT(I-x)Pb(Mg1/2, Nb2/3)O3-xPb-TiO3, PZN-PT/BT Pb(ZN1/2, Nb2/3)O3-x PbTiO3-BaTiO3, (I-x)Pb(ZN1/2, Nb2/3)O3-x(yPbTiO3-(I-y)PbZrO3). In at least one embodiment, the at least one piezoelectric sensor 32 is comprised of a flexible piezoelectric coating (film, paste or paint), such as PVDF or a co-polymer thereof. It will be appreciated to those skilled in the art that recent developments in flexible piezoelectric coatings, (such as U.S. Pat. No. 10,079,336) provide a piezoelectric material 40 that can be applied on a variety of substrates. Of course, other flexible piezoelectric coatings may be utilized in at least one embodiment of the present invention.

As also illustrated in FIG. 2A, in at least one embodiment, multiple layers of piezoelectric material 40 may be stacked on one another, with electrodes 42 positioned therebetween. In at least one further embodiment, as illustrated in FIG. 2B, multiple piezoelectric sensors 32 may be positioned in a side-by-side arrangement. Another benefit with simultaneously screen-printing the at least one piezoelectric sensor 32 and the signal traces onto the resilient substrate 38 is the ability to integrate additional resources such as the EP module 26 and/or the NIRS module 28, as discussed further below. Thus, the sizes, shapes, dimensions, configurations and quantities of each of the at least one piezoelectric sensor 32 and corresponding resilient substrate 38 as depicted in the drawings (and as described herein) are merely exemplary. In further embodiments, each of the at least one piezoelectric sensor 32 and corresponding resilient substrate 38 may take on any other size, shape, dimensions, configurations and/or quantities, now known or later developed, so long as the apparatus 20 is capable of substantially carrying out the functionality described herein. In still further embodiments, any other technique (or combinations of techniques) for positioning the at least one piezoelectric sensor 32 onto resilient substrate 38, now known or later developed, may be substituted.

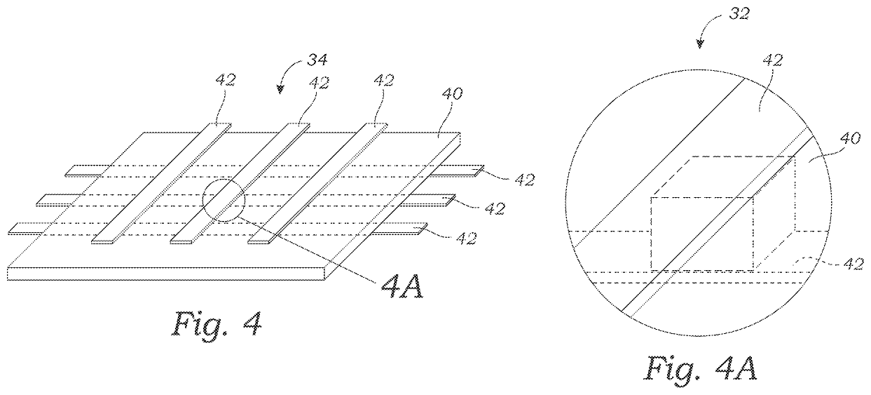

In at least one further embodiment, the piezoelectric material 40 may be sandwiched between a plurality of electrodes 42 which are arranged in a row-column configuration so as to form quasi-two-dimensional arrays. An example of such an embodiment is illustrated in FIGS. 4 and 4A, in which the electrodes 42 are arranged in a 3×3 matrix, and the piezoelectric material 40 is positioned substantially between electrodes 42 in the areas where said electrodes 42 overlap (as illustrated in FIG. 4A).

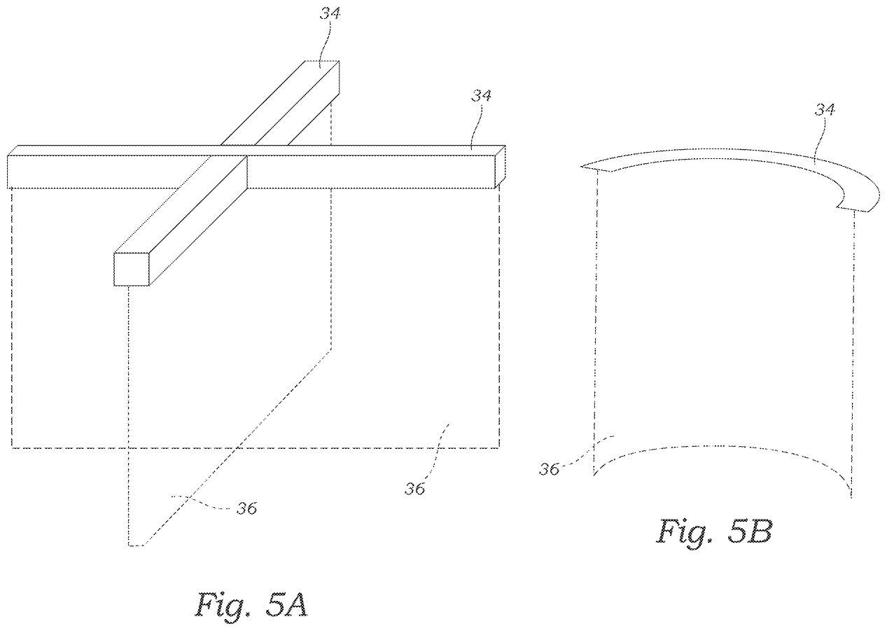

In at least one embodiment, the at least one ultrasound transducer 30 is configured to perform B-mode scans (or “B-scans”) of the target site 24. However, in further embodiments, the at least one ultrasound transducer 30 may be configured to perform other types of scans, now known or later developed, including but not limited to A-mode (or “amplitude mode”), C-mode, M-mode (or “motion mode”), Doppler mode, Pulse inversion mode, Harmonic mode, etc. The positioning of the at least one ultrasound transducer 30 is dependent upon the portion of the user's body that requires an at least one ultrasound image. Additionally, as mentioned above, in at least one embodiment, the at least one ultrasound transducer 30 comprises a plurality of piezoelectric sensors 32 configured as an at least one array 34. In at least one such embodiment, the at least one array 34 may be arranged in a variety of configurations. For example, as illustrated in the diagram of FIG. 5A, two or more linear arrays 34 may be arranged so as to acquire orthogonal cross-sections of the target site 24. As another example, as illustrated in the diagram of FIG. 5B, the at least one array 34 may be configured as a curve (rather than linear). As yet another example, the at least one array 34 may be configured as a convex curve, thereby providing a relatively wider field of view. Additionally, the size of the at least one array 34 is dependent, at least in part, on the specific context in which the apparatus 20 is to be used. As mentioned above, the quantity of piezoelectric sensors 32 in a given array 34 ranges between 2 and 256 in at least one embodiment. However, in further embodiments, any other quantities of piezoelectric sensors 32 may be utilized. Thus, the sizes, shapes, dimensions, configurations and quantities of the at least one array 34 as depicted in the drawings (and as described herein) are merely exemplary. In further embodiments, the at least one array 34 may take on any other size, shape, dimensions, configurations and/or quantities, now known or later developed, so long as the apparatus 20 is capable of substantially carrying out the functionality described herein.

In at least one embodiment, where the at least one ultrasound transducer 30 is configured to perform B-mode scans of the target site and comprises a plurality of piezoelectric sensors 32 configured as an at least one linear array 34, a subset of adjacent or contiguous piezoelectric sensors 32 are configured for being simultaneously excited/activated at any given time. The signals of each piezoelectric sensor 32 of the subset can be identical or present specific time delays to provide focusing or beam steering, while different signal amplitudes on each piezoelectric sensor 32 of the subset may be applied to achieve apodization. Multi-line acquisition techniques may be used to improve frame rate of the resulting at least one ultrasound image. In still further embodiments, multi-line transmission may be utilized in order to further improve frame rate, including the simultaneous excitation of multiple ultrasound beams of either the same or different frequencies. Additionally, in at least one embodiment harmonic imaging is implemented to improve image resolution.

In at least one embodiment, as illustrated in FIG. 1, a bottom surface 44 of the at least one piezoelectric sensor 32 provides an at least one matching layer 46 configured for providing acoustic impedance adaption. When an acoustic wave 36 encounters a boundary between two layers having a relatively large variance in their respective acoustic impedances, the acoustic wave 36 is reflected at the boundary. Thus, in at least one embodiment, using a plurality of matching layers 46 enables the acoustic impedance of each matching layer 46 to be varied gradually to minimize reflections. In at least one such embodiment, the at least one matching layer 46 (or at least a bottom most one of the at least one matching layer 46) is configured for selectively adhering the at least one ultrasound transducer 30 to the target site 24—either directly (i.e., adhering to the user's skin) or indirectly (i.e., adhering to a garment or other material that, in turn, is in contact with the user's skin). In at least one embodiment, the quantity of matching layers 46 is dependent (at least in part) on the characteristics of the at least one piezoelectric sensor 32. In general, it has been found that a higher quantity of matching layers 46 results in relatively better adaption (i.e., less energy is reflected to the at least one ultrasound transducer 30) and broadband operation, which improves the axial resolution of the at least one ultrasound image). In at least one embodiment, where the at least one piezoelectric sensor 32 is screen-printed onto the corresponding at least one resilient substrate 38, the at least one resilient substrate 38 itself may be configured to function as a matching layer 46. In at least one embodiment, similar to the at least one resilient substrate 38, the at least one matching layer 46 is comprised of a material that is flexible and/or stretchable. For example, in at least one such embodiment, the material is a silicone adhesive gel. In further embodiments, the material is at least one of rubber, silicone, thermoplastic elastomers or other polymeric material such as polyester, polyethylene terephthalate, polyethylene naphthalate, polycarbonate, polystyrene, polyacryl, polyether sulfone, etc. Additionally, in at least one embodiment, where the at least one matching layer 46 is configured for being in direct contact with the user's skin, the at least one matching layer 46 is biocompatible, latex-free, non-toxic, and non-allergenic. In still further embodiments, the at least one matching layer 46 may comprise any other appropriate materials (or combinations of materials) having flexible and/or stretchable characteristics, now known or later developed, capable of allowing the at least one matching layer 46 to substantially carry out the functionality described herein.

In at least one embodiment, where the at least one matching layer 46 is comprised of polymeric materials, the acoustic impedance of the polymeric materials may be increased by the incorporation of one or more fillers. Suitable fillers include, but are not limited to, PZT, tungsten, alumina, silica glass, tungsten carbide, titanium, glass powder and the like, with glass powder being preferred. In at least one such embodiment, the size of the filler particles are in the range of approximately 0.1-50 microns and preferably from approximately 0.5-5 microns. The amount of filler employed will be that amount necessary to impart the desired acoustic impedance. Normally, from about 2 to about 50 percent filler by volume and preferably from about 5 to about 30 percent filler by volume is employed. A preferred polymeric material is silicone rubber.

In at least one embodiment, as illustrated in FIG. 1, the ultrasound module 22 further provides a coupling layer 48 positioned in contact with a bottom surface 50 of the at least one matching layer 46 (or the bottom most one of the at least one matching layer 46) and configured for selectively adhering the ultrasound module 22 (and, in turn, the apparatus 20) to the target site 24—either directly (i.e., adhering to the user's skin) or indirectly (i.e., adhering to a garment or other material that, in turn, is in contact with the user's skin). In at least one embodiment, the coupling layer 48 comprises a sonolucent silicone gel or other adhesive material capable of transmitting ultrasound signals between the ultrasound module 22 and the target site 24. By “sonolucent,” it is meant that the gel is capable of transmitting ultrasound pulses without introducing significant interference or attenuation, such that an acceptable acoustic response can be obtained from the target site 24. Thus, the coupling layer 48 materials may be selected for their ability to provide a robust and void-free contact between the ultrasound module 22 and the adjacent target site 24. The acoustic impedance of the coupling layer 48 should be close to that of the adjacent target site 24 to provide impedance matching. In at least one embodiment, the coupling layer 48 is part of the matching layer 46 and its impedance is selected according to the design guidelines of such matching layer 46. In at least one embodiment, where the at least one piezoelectric sensor 32 is screen-printed onto the corresponding at least one resilient substrate 38, the at least one resilient substrate 38 itself may be configured to function as the coupling layer 48. Additionally, in at least one embodiment, the coupling layer 48 provides a temporary backing configured for being peeled off prior to the coupling layer 48 being adhered to the target site 24. In at least one embodiment, the coupling layer 48 has a thickness of approximately 100 to 500 micrometers (e.g., 100, 200, 300, 400, 500 micrometers or some range therebetween). However, in further embodiments, the coupling layer 48 may have any other thickness, so long as the apparatus 20 is capable of substantially carrying out the functionality described herein. Additionally, in at least one embodiment, where the at least one coupling layer 48 is configured for being in direct contact with the user's skin, the at least one coupling layer 48 is biocompatible, latex-free, non-toxic, and non-allergenic. In still further embodiments, the at least one coupling layer 48 may comprise any other appropriate materials (or combinations of materials) having the above described characteristics, now known or later developed, capable of allowing the at least one coupling layer 48 to substantially carry out the functionality described herein.

In at least one embodiment, as illustrated in FIG. 1, the ultrasound transducer 30 further provides an at least one backing layer 52 positioned in contact with the at least one piezoelectric sensor 32 opposite the corresponding side at which energy is intended to be radiated (i.e., where the target site 24 is located), such that the at least one piezoelectric sensor 32 is substantially sandwiched between the at least one backing layer 52 and the target site 24. The at least one backing layer 52 is configured for absorbing any ultrasound waves radiated by the at least one piezoelectric sensor 32 that is not directed toward the target site 24, thus preventing any reverberations and/or resonances that would otherwise decrease the bandwidth of the emitted pulses from the at least one piezoelectric sensor 32. In at least one embodiment, where the at least one piezoelectric sensor 32 is screen-printed onto the corresponding at least one resilient substrate 38, the at least one resilient substrate 38 itself may be configured to function as the backing layer 52. Accordingly, in at least one embodiment, the at least one backing layer 52 is made of a material having an acoustic impedance close to the at least one piezoelectric sensor 32 and having a relatively high damping coefficient. In such embodiments, because the acoustic impedance of the at least one backing layer 52 is similar to that of the at least one piezoelectric sensor 32 and because of the absorption of the material of the at least one backing layer 52, most of the backward transmitted wave quickly attenuates and become heat, and only a very small portion may bounce back. In at least one embodiment, the at least one backing layer 52 is constructed out of at least one of tungsten-loaded epoxy, pyrolytic, brass, carbon, etc. In still further embodiments, the at least one backing layer 52 may comprise any other appropriate materials (or combinations of materials), now known or later developed, capable of allowing the at least one backing layer 52 to substantially carry out the functionality described herein.

With continued reference to FIG. 1, in at least one embodiment, the ultrasound module 22 further provides a pair of conductive layers 54 positioned for substantially sandwiching the at least one piezoelectric sensor 32 therebetween, such that the conductive layers 54 integrate the electrodes 42 of each piezoelectric sensor 32 as well as the traces that interconnect them to the electronics system (hereinafter referred to as the “microelectronics module” 56, as discussed further below) associated with the ultrasound module 22. In at least one embodiment, the conductive layers 54 each comprises a thin metal film such as aluminum, copper, gold, molybdenum, iridium, magnesium, silver, lithium fluoride and alloys thereof, or a non-metal material. Additionally, in at least one embodiment, the thickness of each conductive layer 54 is typically about 200 μm or less (e.g., about 200, 180, 160, 140, 120, 100, 90, 80, 70, 60, 50, 40, 30 μm or less). Preferably, the thickness of each conductive layer 54 is less than 10 μm (e.g., about 10, 9, 8, 7, 6, 5, 4, 3, 2, 1, 0.8, 0.6, 0.4, 0.2 μm or less or some range therebetween). Additionally, in at least one embodiment, the conductive layers 54 are flexible. In at least one such embodiment, the conductive layers 54 are constructed out of transparent conductive polymer materials, such as indium tin oxide (ITO), fluorine-doped tin oxide (FTO), ZnO—Ga2O3, ZnO—Al2O3, SnO2-Sb2O3, and polythiophene. In addition, the conductive layers 54 may be comprised of silver or copper grids or bushbars plated on a transparent substrate or silver nanowires or nanoparticles deposited on a substrate with a poly(3,4-ethylenedioxythiophene) poly(styrenesulfonate) (PEDOT:PSS) coating. Additional conductive polymer layers may be added to improve conductivity. In at least one embodiment, the conductive layers 54 may be carbon-based, for example, carbon nanotubes (“CNT”), carbon nanowires, or graphene, and the like. One preferred conductive layer 54 (electrically conductive and transparent for infrared radiation) comprises graphene. While one or two layers of graphene is preferred, the conductive layers 54 may each comprise about 1 to 20 layers of graphene (e.g., 1, 2, 3, 4, 5, 6, 7, 8, 9, 10, 11, 12, 13, 14, 15, 16, 17, 18, 19, 20 layers or some range therebetween). In at least one embodiment, the conductive layers 54 may comprise several internal conductive layers 54 separated by insulating materials in order to manage a high number of tracks.

With continued reference to FIG. 1, in at least one embodiment, the ultrasound transducer 30 further provides a pair of encapsulation layers 58 positioned for substantially sandwiching the pair of conductive layers 54 (and, in turn, the at least one piezoelectric sensor 32) therebetween, such that the encapsulation layers 58 are configured for isolating the at least one piezoelectric sensor 32 from the surrounding environment. In at least one embodiment, the encapsulation layers 58 are substantially impermeable to moisture and oxygen. In general, the moisture and oxygen sensitive components of the apparatus 20 should be enclosed by materials having gas permeation properties. The encapsulation layers 58 preferably achieve low water vapor permeation rates of 10-4 g/ni²/day or less, 10-5 g/m²/day or less, and even more preferably about 10-6 g/ni²/day or less. In at least one embodiment, the encapsulation layers 58 are constructed out of glass or a plastic, for example. In at least one embodiment, the encapsulation layers 58 are comprised of a material that is flexible and/or stretchable. For example, in at least one such embodiment, the material is comprised of at least one of a silicon-based material, rubber, thermoplastic elastomers, polymeric materials, foils (such as those mixed with epoxy), and various fabrics. Ideally, substrates in direct contact with organic layers will have exceptional barrier capabilities that withstand heat, offer flexibility, have sustained reliability and can be mass produced.

As mentioned above, in at least one embodiment, the apparatus 20 further provides an electrophysiological (“EP”) module 26 configured for detecting bioelectric signals of the target site 24. In at least one such embodiment, the at least one EP module 26 is configured as a surface electromyography (“sEMG”) sensor in order to detect the electric potential generated by muscles fibers (myocyte). The frequency range of the EMG amplitude is 20 μV-5 μV; however, other amplitudes may be substituted in further embodiments. When more muscles fibers are recruited to sustain constant loads or to support an increase in load, the amplitude of the sEMG signal increases. In at least one embodiment, the sEMG signal reflects muscular activation driven by the motoneuron, and can be non-invasively collected from skin surface. As an effective tool, sEMG sensor can be used in the diagnosis of neuromuscular diseases, assessment of muscle fatigue and human-machine interface for prosthetic manipulation. In at least one such embodiment, when combining the various modalities of the sEMG sensor with the functionality of the ultrasound module 22, the apparatus 20 is capable of being used in a wide variety of contexts, including but not limited to: exercise and training; identifying muscles, tendons and other soft tissues injuries; identifying myoelectric manifestations of fatigue; assessing EMG signal modifications in pathologies; evaluating motor coordination and treatment efficacy; identifying neurological diseases; identifying disuse, immobility, and physical inactivity; and measuring neuromuscular alteration due to age. In at least one embodiment, the apparatus 20 is configured for being tightly/closely engaged with the user's skin (so as to eliminate or at least minimize movement artifacts or the displacement of the apparatus 20), substantially over top of the muscle of interest.

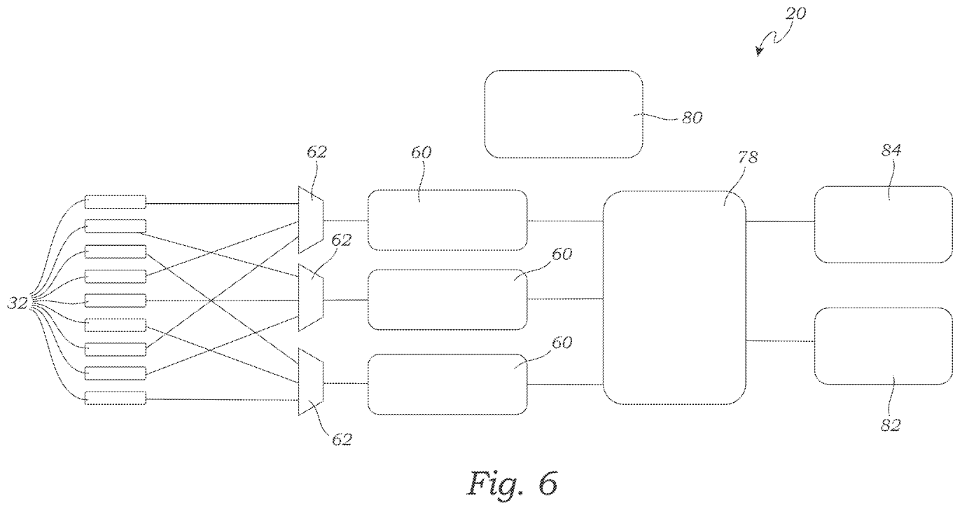

In at least one embodiment, as illustrated in FIG. 6, the apparatus 20 further provides an at least one ultrasound transceiver 60 in electrical communication with the at least one piezoelectric sensor 32 of the ultrasound module 22. In at least one such embodiment, the apparatus 20 provides a relatively greater quantity of piezoelectric sensors 32 than ultrasound transceivers 60, such that the at least one ultrasound transceiver 60 is in electrical communication with a plurality of piezoelectric sensors 32. In at least one such embodiment, the apparatus 20 provides an at least one analog bidirectional multiplexer 62 in electrical communication with the at least one ultrasound transceiver 60 and the corresponding plurality of piezoelectric sensors 32. An exemplary configuration of multiplexers 62 is illustrated in the schematic view of FIG. 6. However, it should be noted that the configurations and quantities of the at least one multiplexer 62 as depicted in the drawings (and as described herein) are merely exemplary. In further embodiments, the at least one multiplexer 62 may take on any other configurations (relative to the at least one ultrasound transceiver 60 and the corresponding plurality of piezoelectric sensors 32) and/or quantities, now known or later developed, so long as the apparatus 20 is capable of substantially carrying out the functionality described herein. In at least one embodiment, where the apparatus 20 provides a plurality of multiplexers 62, the multiplexers 62 are arranged in a multilayer configuration, with the signal traces between in each layer varying depending on the quantities of multiplexers 62 and piezoelectric sensors 32. For example, in at least one such embodiment, the quantity of layers is equal to the quantity of multiplexers 62; while in at least one further such embodiment, the quantity of layers is equal to the quotient of the quantity of piezoelectric sensors 32 divided by the quantity of multiplexers 62. In still further embodiments, any other quantity of layers, and any other arrangement of signal traces between said layers, may be substituted, so long as the apparatus 20 is capable of substantially carrying out the functionality described herein.

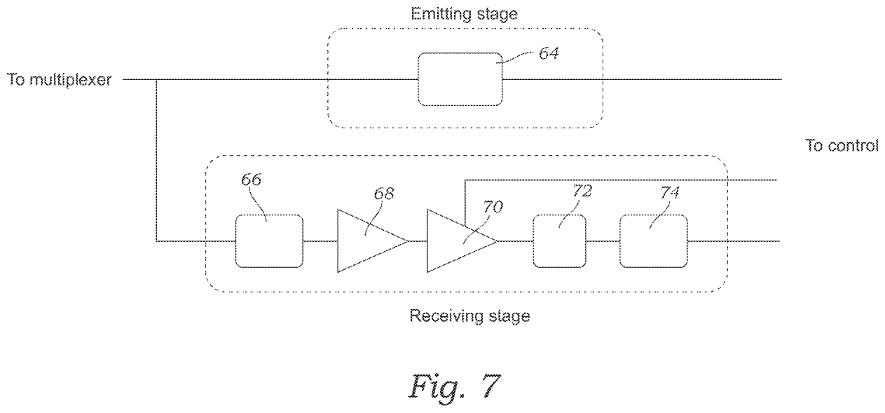

In at least one embodiment, as illustrated in the simplified schematic of FIG. 7, the at least one ultrasound transceiver 60 is itself comprised of a pulser 64, a transmission/reception switch (“T/R switch”) 66, a low noise amplifier (“LNA”) 68, a variable gain amplifier (“VGA”) 70, a low-pass filter (“LPF”) 72, and an analog-to-digital converter (“ADC”) 74. In a bit more detail, in at least one such embodiment, the at least one ultrasound transceiver 60 is capable of emitting ultrasound pulses with several discrete levels to provide amplitude apodization. In still further embodiments, the at least one ultrasound transceiver 60 may emit pulses with any waveform, and subsequently perform an accurate amplitude apodization which would even include the emission of limited diffraction beams such as the zeroth-order Bessel beam.

Referring again to FIG. 6, in at least one embodiment, the apparatus 20 further provides an at least one controller 78 in electrical communication with each of the ultrasound module 22, EP module 26 and/or NIRS module 28. Thus, in such embodiments, the controller 78 is configured for interfacing with and managing each of the ultrasound module 22, EP module 26 and/or NIRS module 28, and processing the at least one ultrasound image of the target site 24. In at least one such embodiment, processing may include image reconstruction and/or data compression. In at least one alternate embodiment, one or more of the ultrasound module 22, EP module 26 and/or NIRS module 28 provide their own dedicated power supply 80. In at least one embodiment, the controller 78 is also in electrical communication with an at least one transceiver 82 configured for transmitting the at least one ultrasound image, and any data associated therewith, to select external devices such as computing and electrical devices in communication with the apparatus 20. In at least one further embodiment, the at least one transceiver 82 is further configured for receiving select information from such external devices as well. The at least one transceiver 82 may utilize any wired- or wireless-based communication protocol (or combination of protocols) now known or later developed, including but not limited to Wi-Fi and Bluetooth-LE.

Additionally, in at least one embodiment, the controller 78 is in selective communication with an at least one data storage device 84 (either locally or remotely) configured for storing the at least one ultrasound image and any data associated therewith. It should be noted that the term “data storage device” is intended to include any type of electronic storage medium (or combination of storage mediums) now known or later developed, such as local hard drives, RAM, flash memory, secure digital (“SD”) cards, external storage devices, network or cloud storage devices, integrated circuits, etc.

In at least one further embodiment, where the apparatus 20 incorporates further modules (such as the EP module 26 and/or NIRS module 28, for example), the controller 78 is configured for managing any such further modules. Additionally, in embodiments where the apparatus 20 provides the EP module 26, the controller is capable of selectively triggering the ultrasound image acquisition upon detection of bioelectric signal and/or specific value of the electrophysiological parameter in the target site 24, which prevents measurements during irrelevant periods and optimizes the usage of energy intended for ultrasound imaging. Such functionality also allows acquisition at specific instances or when specific events are detected. In at least one such embodiment, the controller 78 is able to optimize the acquisition of ultrasound images of the target site 24 during very fast repetitive movements, even with low acquisition rates—given that the ultrasound image capture process can be synchronized with the periodic movement, the ultrasound images or their lines can be acquired along several periods. In still further embodiments, the controller 78 may be configured to selectively control other aspects and/or functions of the apparatus 20—such as running the various components in a “low power mode” for example. In at least one embodiment, the controller 78 is at least one of a field-programmable gate array (“FPGA”), a digital signal processor (“DSP”), a microcontroller, and a microprocessor.

With continued reference to FIG. 6, in at least one embodiment, the apparatus 20 further provides a power supply 80. The power supply 80 may be any source of power-now known or later developed-capable of providing the necessary power to each of the ultrasound module 22, EP module 26 and/or NIRS module 28, including but not limited to one or more batteries (rechargeable or otherwise), an AC adapter, a DC adapter, etc. In at least one alternate embodiment, one or more of the ultrasound module 22, EP module 26 and/or NIRS module 28 provide their own dedicated power supply 80.

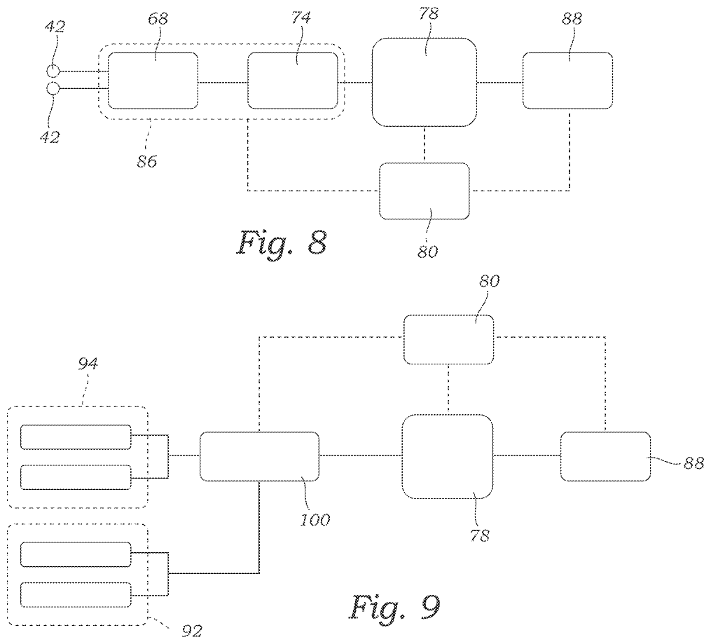

In at least one embodiment, as illustrated in FIG. 8, the EP module 26 is configured as a multi-channel, compact wireless acquisition system, providing an at least one electrode 42, a front-end signal conditioning circuit 86, a power supply 80, a controller 78, and a wireless communication module 88, such as a Bluetooth-LE module. In at least one embodiment, the EP module 26 provides a biocompatible printed-electrode array to capture bioelectric signals. Additionally, in at least one embodiment, as illustrated in FIG. 1, the EP module provides at least one electrode array comprising 32 electrodes 42 or less (e.g., about 32, 30, 28, 26, 24, 22, 20, 18, 16, 14, 12, 10, 8, 6, 4, 2), along with a reference electrode 90. However, in further embodiments, any other quantities of electrodes 42 may be utilized. In at least one embodiment, the reference electrode 90 is positioned between two differential electrodes 42 to avoid asymmetry in bioelectric signals recording, and the inter-electrode spacing is increased with this electrode 42 configuration. However, a small inter-electrode spacing is preferable as it will reduce the amount of crosstalk signal detected from adjacent active muscles. Therefore, the inter-electrode spacing are set to be about 32 to 8 millimeters as a preferred compromise (e.g., about 32, 31, 30, 29, 28, 27, 26, 25, 24, 23, 22, 21, 20, 19, 18, 17, 16, 15, 14, 13, 12, 11, 10, 9, 8 mm, or some range therebetween). However, in further embodiments, any other spacing may be utilized, so long as the apparatus 20 is capable of substantially carrying out the functionality described herein. Additionally, in at least one embodiment, the electrodes 42 have a thickness of about 100 micrometers or less (e.g., about 100, 90, 80, 70, 60, 50, 40, 30, 20, 10, 5 μm or less). However, in further embodiments, the electrodes 42 may have any other thickness, so long as the apparatus 20 is capable of substantially carrying out the functionality described herein. The EP module 26 may optionally include electrodes 42 with different sizes and shapes, for example rectangular, circle, oval, ring or disk-shaped electrodes. In a non-limiting example, the electrode 42 array is characterized in that a disk-shaped conductor and at least one ring conductor concentric with the disk-shaped conductor which are disposed on the substrate in order to capture bioelectric signals and configured to provide different weights to the voltages of the conductors generating multiple outputs, corresponding to different sensitivity-based spatial distributions, configured according to requirements for the capture of the bioelectric potentials to be measured. In at least one embodiment, the EP module 26 further provides a coupling layer 48 positioned in contact with a bottom surface of the at least one electrode 42 (or the bottom most one of the at least one electrode 42 array) and configured for selectively adhering the EP module 26 (and, in turn, the apparatus 20) to the target site 24—either directly (i.e., adhering to the user's skin) or indirectly (i.e., adhering to a garment or other material that, in turn, is in contact with the user's skin). In at least one embodiment, the coupling layer 48 comprises a gel, (e.g., a hydrogel with adhesive properties). The hydrogel may be electrically conductive and capable of transmitting bioelectric signals between the target site 24 and the EP module 26.

In at least one embodiment, the electrodes 42 and conductive tracks of the EP module 26 are comprised of conductive metallic inks/pastes produced by using metallic nanoparticles, metal-organic complexes, or metallic salts as precursors (mostly silver-based), conductive polymers, as their conductivity is typically lower than their metallic counterparts but their adhesion and mechanical stability are better, and they do not usually require post-treatment steps. Alternatively, dispersions of graphene or CNT's can also be used in printing to create conductive electrodes 42 and/or tracks (conductor patterns). In at least one such embodiment, for example, the electrodes 42 and conductive tracks of the EP module 26 comprise silver polymer paste, stretchable silver conductor paste, medical grade electrically conductive Ag/AgCl ink, or poly(3,4-ethylenedioxythiophene)-poly (styrenesulfonate) due to their flexible processing and durable electrical conductivity.

As mentioned above, in at least one embodiment, the apparatus 20 further provides a near-infrared spectroscopy (“NIRS”) module 28 configured for monitoring oxygenation status and/or biochemical measurements of the target site 24. In at least one such embodiment, as illustrated in FIGS. 1 and 9, the at least one NIRS module 28 comprises an at least one photodetector 92 and an at least one near-infrared light emitting diode (“LED”) 94 for muscle oxygenation measurement (muscle oximetry) supported by a substrate.

Human tissues are relatively transparent to light in the near-infrared range between 650-1000 nm. The near-infrared radiation (“NIR”) window, also known as the “optical window”, is the range of wavelengths that has the maximum depth of penetration in tissue. Indeed, because NIR is minimally absorbed by water and hemoglobin, spectra readings can be easily collected from the body surface, and the main absorbers are blood chromophores of oxygenated hemoglobin (HbO2) and deoxygenated hemoglobin (HHb). When the near-infrared light emitted by the LED 94 passes though the tissue, a portion of light is reflected and absorbed, the remaining light is scattered and can be measured by the at least one photodetector 92. The depth of the detected NIRS signal can be controlled by the distance between the LED 94 and photodetector 92. It is generally accepted that, for a source-detector distance of 3 cm, about 1.5 cm (half of the source-detector distance) below the skin surface can be detected through a banana-shaped region. Thus, considering specified anatomies of different muscles, the LED-detector distance can be selected in the range of 2-7 cm for muscle activities detection. However, in further embodiments, any other spacing may be utilized, so long as the apparatus 20 is capable of substantially carrying out the functionality described herein. Moreover, HbO2 and HHb have differential optical absorption properties to the near-infrared light, contracting a muscle changes the amount of near-infrared light that is scattered back to the skin surface, and this change can be detected by the photodetector 92. By using a modified Beer-Lambert law, relative concentration changes of HbO2 and HHb can be calculated and quantified.

Therefore, combining the advantage of the EP module 26 and NIRS module 28, in at least one embodiment, would facilitate the understanding of muscle activities from the view of electrophysiology and metabolism, providing more valuable information for human health and physiology performance. For example, NIRS combined with sEMG sensor has been used to obtain more reliable information for assessing metabolic and neuromuscular activity, suggesting the mechanisms of muscle fatigue or injury. However, individual sEMG and NIRS sensor systems were adopted, which resulted in large size, troublesome data synchronism, cumbersome signal wires and limited channels. Thus, the apparatus 20 having integrated EP and NIRS modules 26 and 28 along the ultrasound module 22, in at least one embodiment, is of great importance to fulfill clinical practical requirements.

In at least one embodiment, one or more of the at least one photodetector 92 may serve to provide a reference signal. For example, the photodetector 92 closest to the near-infrared LED 94 may provide a reference intensity against which the intensities measured by the other photodetectors 92. In this manner, control over and knowledge of the variations in intensity of the signals emitted by the near-infrared LED 94 may be provided, simplifying the NIRS module 28 design and operation. In at least one embodiment, the photodetectors 92 may be spaced 10 millimeters apart between centers of each adjacent photodetector 92 and between the center of the first photodetector 92 of the plurality of further photodetectors 92 and the near-infrared LED 94. In other configurations, smaller spacing may be used to allow for inclusion of a greater number of photodetectors 92, such as a spacing of 8 mm. For example, the photodetectors 92 may be spaced from the near-infrared LED 94 by 8 mm, 16 mm, 24 mm, and 32 mm, respectively. In some configurations, the spacing between neighboring photodetectors 92 may be between 5 mm and 20 mm, less than 5 mm, less than 1 mm or any distance or range of distances within such ranges. In further embodiments, any other spacing may be utilized, so long as the apparatus 20 is capable of substantially carrying out the functionality described herein. In at least one embodiment, the photodetectors 92 are arranged and configured electronically to be operated synchronously with the near-infrared LED 94. In at least one such embodiment, a photometric front-end 100 is utilized to operating the photodetectors 92 and LED 94. In at least one embodiment, the near-infrared LED 94 may include a thin light source that may comprise, for example, OLEDs or printable LEDs (organic or inorganic). In at least one such embodiment, the light source comprises a flexible light emitter located between two conductive layers 54 (i.e., electrodes) comprising an anode and a cathode, wherein the flexible light emitter emits light in response to an electric current applied to the anode and cathode. One typical light source uses a transparent substrate, a transparent anode, a flexible light emitter, and a reflective cathode. Light generated from the flexible light emitter is emitted through the transparent anode and transparent substrate. This is commonly referred to as a bottom-emitting light source. By way of example, in at least one such embodiment, a plurality of photodetectors 92 are arranged substantially linearly on a path that originates from the location of the near-infrared LED 94 for measurement of light signals at varying locations from the near-infrared LED 94. In a preferred configuration, at least two photodetectors 92 are used to measure the intensity of light signals for at least two different distances from the LED 94, to provide an improved fitting of measured signals as a function of distance with a model used to provide any one or more of oxy(+myo)hemoglobin, (O₂Hb), deoxyhemo(+myo)globin (HHb), total hemo(+myo)globin (tHb) or muscle oxygen saturation (SmO₂) based on the measured intensities.

In at least one embodiment, the conductive layers 54 may comprise a shared electrode such that the same conductive layer 54 serves as a common cathode or as a common anode for the ultrasound module 22, the EP module 26 and/or the NIRS module 28. The anode for the EP module 26 and/or the NIRS module 28 comprises, for example, a transparent conductive oxide (TCO), such as, but not limited to, indium tin oxide (ITO), zinc oxide (ZnO), and the like. In practice conductive layers 54 will include a network of tracks connecting these components to their associated electronic systems. Conductive layers 54 additional to those containing the anode or cathode of the ultrasound module 22, EP module 26 or NIRS module 28 may be included to allow proper routing of all tracks. Moreover conductive layers 54 having a continuous conductive plane may be considered to allow the implementation of impedance-controlled traces (such as microstrip or strip line) and/or provide electromagnetic shielding. Conductive planes or traces may be considered to collect heat generated by any element of the apparatus 20 and conduct it to at least one heat sink where such a heat may be safely transferred to the ambient environment.

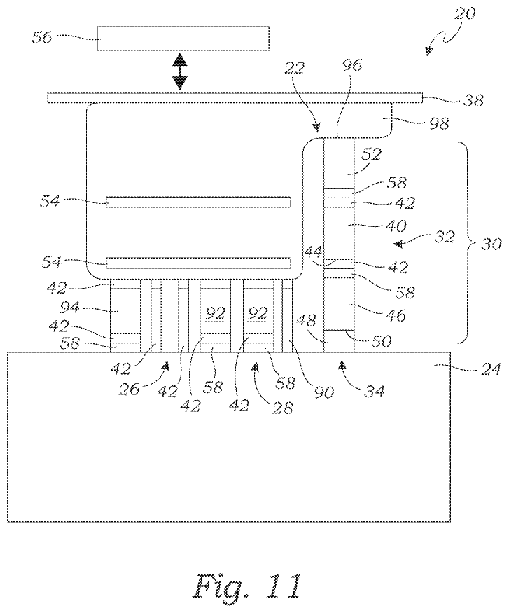

In at least one embodiment, one or more of the electronics system, such as, but not limited to, ultrasound transceiver 60, analog bidirectional multiplexer 62, controller 78, transceiver 82, power supply 80, front-end signal conditioning circuit 86, photometric front-end 100, wireless communication module 88, data storage device 84, and the like, may be contained within at least one microelectronics module 56 (FIG. 1) positioned in contact with a top surface 96 of the at least one backing layer 52. In at least one further embodiment, the microelectronics module 56 is positioned within a covering 98 positioned on top of and in electrical contact with the various components of the apparatus 20. In still further embodiments, the microelectronics module 56 may be positioned elsewhere on or relative to the apparatus 20. For example, in at least one such further embodiment, as illustrated in FIG. 11, the microelectronics module 56 may be positioned external to or separate from the other components of the apparatus 20, with each of the at least one ultrasound module 22, EP module 26 and/or NIRS module 28 being in electrical communication with the microelectronics module 56. Such embodiments allow for fabricating the microelectronics module 56 through traditional and relatively reliable processes while also increasing the modularity of the apparatus 20. In still further such embodiments, each of the at least one ultrasound module 22, EP module 26 and/or NIRS module 28 may be implemented together in a single flexible patch (as discussed further below) or separately-dependent at least in part on the technology required in a given use case and the location of the target site 24.

In at least one embodiment, the covering 98 is constructed out of a transparent or semi-transparent material. However, in further embodiments, the covering 98 may be constructed out of an opaque material. The covering 98 may provide comfort for a user, particularly if the user is engaged in physical activity. The covering 98 may provide protection to the various components of the apparatus 20, keeping dirt and fluid off of the components and providing a cushion to protect the apparatus 20 from impact. The covering 98 may additionally improve the heat transfer between any component of the apparatus 20 and the surrounding environment, provided a reasonably low heat resistivity of the material.

It should be noted that the configuration and arrangement of the various components of the apparatus 20 (including the relative positioning of the components of each of the ultrasound module 22, EP module 26 and/or NIRS module 28) as depicted in the drawings is merely exemplary. Accordingly, in further embodiments, the various components may take on any other configurations and arrangements, now known or later developed, so long as the apparatus 20 is capable of substantially carrying out the functionality described herein.

As also illustrated in FIG. 1, in at least one embodiment, the various components of the apparatus 20 described above are configured as a self-contained wearable patch-which can either be adhesively secured to the user's skin (or to a garment in direct contact with the user's skin), or alternatively secured or otherwise integrated into the fabric of a garment that is in direct contact with the user's skin. In each such instance, the apparatus 20 is configured as a wearable, flexible solution for providing remote ambulatory monitoring of the target site. Thus, in embodiments where the apparatus 20 is utilized in the context of MSK ultrasounds (which are normally used to generate ultrasound images of muscles, tendons, ligaments and joints throughout the body, helping to diagnose sprains, strains, tears, and other soft tissue conditions), the apparatus 20 will allow real-time data collection in the sports medicine and real-time healthcare monitoring domains, while also being useful in a multitude of advanced applications, including human-machine interfaces, advanced prosthetic technologies (bionics), electronic skins, wearable consumer electronics and soft robotics, to name a few. Furthermore, by incorporating each of the ultrasound module 22, EP module 26 and NIRS module 28 in at least one embodiment, the apparatus 20 is capable of functioning as a novel multi-modal “3-in-1” system (or at least a “2-in-1” system, where only one of the EP module 26 or NIRS module 28 are incorporated with the ultrasound module 22) that simultaneously acquires ultrasonic imaging, bioelectric signals and oxygenation status and/or biochemical measurements-including but not limited to sonomyography (“SMG”), electromyography (“EMG”), electrocardiogramcephalography (“EEG”), galvanic skin response (“GSR”), photoplethysmography (“PPG”), arterial oxygen saturation (“Sp02”), oxy(+myo)hemoglobin, (O₂Hb), deoxyhemo(+myo)globin (HHb), total hemo(+myo)globin (tHb), muscle oxygen saturation (SmO₂), muscle activity, emotions, arterial saturation of carbon monoxide (“SpCO”) and blood carbon dioxide (“CO2”), blood pressure (“BP”), respiration, such as respiration frequency (“RF”) and/or respiration volume (“RV”), heart rate (“HR”) and/or heart rate variability (“HRV”), pulse, bioimpedance, and temperature, such as skin temperature (“ST”), and/or core body temperature—in various biomedical and clinical applications.

Aspects of the present specification may also be described as the following embodiments:

1. A wearable ultrasound apparatus positionable on a target site of a user's body and comprising: an at least one ultrasound module configured for obtaining an at least one ultrasound image of the target site, the ultrasound module comprising: an at least one ultrasound transducer positioned on an at least one resilient substrate, the at least one ultrasound transducer comprising an at least one sensor; an at least one conductive layer positioned in electrical communication with the at least one sensor; and an at least one ultrasound transceiver in electrical communication with the at least one sensor; an at least one electrophysiological (“EP”) module positioned on the at least one resilient substrate and configured for detecting bioelectric signals of the target site; an at least one controller in electrical communication with each of the ultrasound module and EP module via the conductive layers, the at least one controller configured for selectively causing the ultrasound module to obtain at least one ultrasound image of the target site upon detection of bioelectric signal in the target site via the EP module.

2. The wearable ultrasound apparatus according to embodiment 1, wherein the at least one sensor is at least one of a piezoelectric sensor or a microelectromechanical (“MEM”) sensor.

3. The wearable ultrasound apparatus according to embodiments 1-2, wherein the at least one sensor is a piezoelectric sensor comprising a piezoelectric material sandwiched between two or more electrodes.

4. The wearable ultrasound apparatus according to embodiments 1-3, wherein the at least one ultrasound module comprises a pair of conductive layers positioned for substantially sandwiching the at least one piezoelectric sensor therebetween.

5. The wearable ultrasound apparatus according to embodiments 1-4, wherein the at least one ultrasound module further comprises an at least one matching layer positioned on a bottom surface of the at least one sensor and configured for providing acoustic impedance adaption.

6. The wearable ultrasound apparatus according to embodiments 1-5, wherein the at least one ultrasound module further comprises an at least one encapsulation layer positioned to isolate the at least one conductive layer from the surrounding environment.

7. The wearable ultrasound apparatus according to embodiments 1-6, wherein the at least one ultrasound module further comprises a pair of encapsulation layers positioned for substantially sandwiching the at least one conductive layer therebetween.

8. The wearable ultrasound apparatus according to embodiments 1-7, wherein the at least one ultrasound transducer is configured for operating in the range of 7-14 MHz for superficial scans and 2-6 MHz for deeper targets.

9. The wearable ultrasound apparatus according to embodiments 1-8, wherein the at least one ultrasound transducer is configured for operating in a pulse-echo configuration.

10. The wearable ultrasound apparatus according to embodiments 1-9, wherein each of the at least one piezoelectric sensor has a width that is relatively less than a pitch thereof.

11. The wearable ultrasound apparatus according to embodiments 1-10, wherein the at least one ultrasound transducer comprises a plurality of adjacently arranged sensors configured as an at least one array.

12. The wearable ultrasound apparatus according to embodiments 1-11, wherein a distance between centers of two contiguous piezoelectric sensors of an array is less than approximately 0.5λ for phased array operation and approximately 0.75λ-3λ for linear array operation, where λ=c/f, with λ being the wavelength of the ultrasound signal with frequency f and longitudinal sound speed c≈1500 m/s.

13. The wearable ultrasound apparatus according to embodiments 1-12, wherein contiguous piezoelectric sensors of an array are separated by a small kerf so as to provide acoustic inter-element isolation to each of the piezoelectric sensors.

14. The wearable ultrasound apparatus according to embodiments 1-13, wherein the at least one ultrasound transducer comprises a plurality of arrays positioned in a side-by-side arrangement.

15. The wearable ultrasound apparatus according to embodiments 1-14, wherein the at least one ultrasound transducer comprises a plurality of arrays arranged so as to acquire orthogonal cross-sections of the target site.

16. The wearable ultrasound apparatus according to embodiments 1-15, wherein the at least one array is configured as a curve.

17. The wearable ultrasound apparatus according to embodiments 1-16, wherein a subset of contiguous sensors are configured for being simultaneously activated on demand.

18. The wearable ultrasound apparatus according to embodiments 1-17, wherein a plurality of sensors are sandwiched between a plurality of electrodes arranged in a row-column configuration so as to form an at least one quasi-two-dimensional array.

19. The wearable ultrasound apparatus according to embodiments 1-18, wherein a bottom most one of the at least one matching layer is configured for selectively adhering the corresponding at least one ultrasound transducer to the target site.

20. The wearable ultrasound apparatus according to embodiments 1-19, wherein the at least one matching layer is constructed out of at least one of a silicone adhesive gel, rubber, silicone, thermoplastic elastomers, and polymeric materials.

21. The wearable ultrasound apparatus according to embodiments 1-20, wherein the at least one matching layer is further constructed out of a material that is biocompatible, latex-free, non-toxic, and non-allergenic.

22. The wearable ultrasound apparatus according to embodiments 1-21, wherein the at least one matching layer is constructed out of polymeric materials along with an at least one filler comprising at least one of PZT, tungsten, alumina, silica glass, tungsten carbide, titanium and glass powder, said at least one filler configured for increasing the acoustic impedance of the polymeric materials.

23. The wearable ultrasound apparatus according to embodiments 1-22, wherein the ultrasound module further comprises a coupling layer positioned in contact with a bottom surface of a bottom most one of the at least one ultrasound transducer and configured for selectively adhering the ultrasound module to the target site.

24. The wearable ultrasound apparatus according to embodiments 1-23, wherein the coupling layer positioned in contact with a bottom surface of a bottom most one of the at least one matching layer.

25. The wearable ultrasound apparatus according to embodiments 1-24, wherein the coupling layer comprises a sonolucent silicone gel or other adhesive material capable of transmitting ultrasound signals between the ultrasound module and the target site.

26. The wearable ultrasound apparatus according to embodiments 1-25, wherein the coupling layer is further constructed out of a material that is biocompatible, latex-free, non-toxic, and non-allergenic.

27. The wearable ultrasound apparatus according to embodiments 1-26, wherein the coupling layer has an acoustic impedance that approximates an acoustic impedance of the target site so as to provide impedance matching.

28. The wearable ultrasound apparatus according to embodiments 1-27, wherein the ultrasound module further comprises an at least one backing layer positioned in contact with the side of the at least one sensor furthest from the target site, the at least one backing layer configured for absorbing any ultrasound waves radiated by the at least one sensor that are not directed toward the target site.

29. The wearable ultrasound apparatus according to embodiments 1-28, wherein the at least one backing layer has an acoustic impedance that approximates an acoustic impedance of the at least one sensor.

30. The wearable ultrasound apparatus according to embodiments 1-29, wherein the at least one backing layer is constructed out of at least one of tungsten-loaded epoxy, pyrolytic, brass, and carbon.

31. The wearable ultrasound apparatus according to embodiments 1-30, wherein the at least one conductive layer is constructed out of at least one of conductive polymer materials, carbon, graphene, aluminum, copper, gold, molybdenum, iridium, magnesium, silver, lithium fluoride and alloys thereof.

32. The wearable ultrasound apparatus according to embodiments 1-31, wherein the at least one encapsulation layer is constructed out of at least one of glass and plastic.

33. The wearable ultrasound apparatus according to embodiments 1-32, wherein the at least one encapsulation layer is substantially impermeable to moisture and oxygen.

34. The wearable ultrasound apparatus according to embodiments 1-33, wherein the apparatus comprises a relatively greater quantity of sensors than ultrasound transceivers, such that the at least one ultrasound transceiver is in electrical communication with a plurality of sensors.

35. The wearable ultrasound apparatus according to embodiments 1-34, further comprising an at least one bidirectional multiplexer in electrical communication with the at least one ultrasound transceiver and the corresponding plurality of sensors.

36. The wearable ultrasound apparatus according to embodiments 1-35, wherein the EP module comprises an at least one electrode array and an at least one reference electrode.

37. The wearable ultrasound apparatus according to embodiments 1-36, wherein the at least one ultrasound transceiver comprises a pulser, a transmission/reception switch (“T/R switch”), a low noise amplifier (“LNA”), a variable gain amplifier (“VGA”), and a low-pass filter (“LPF”).

38. The wearable ultrasound apparatus according to embodiments 1-37, further comprising an at least one transceiver in electrical communication with the at least one controller and configured for communicating with select external devices.

39. The wearable ultrasound apparatus according to embodiments 1-38, wherein the EP module comprises an at least one electrode, a front-end signal conditioning circuit, a controller, and a communication module.

40. The wearable ultrasound apparatus according to embodiments 1-39, wherein the at least one resilient substrate is constructed out of at least one of a silicon-based material, rubber, thermoplastic elastomers, polymeric materials, foils, and various fabrics.

41. The wearable ultrasound apparatus according to embodiments 1-40, wherein the at least one resilient substrate is further constructed out of a material that is transparent, flexible, and conformable in nature.

42. The wearable ultrasound apparatus according to embodiments 1-41, wherein the at least one resilient substrate is further constructed out of a material that is biocompatible, latex-free, non-toxic, and non-allergenic.

43. The wearable ultrasound apparatus according to embodiments 1-42, wherein the at least one resilient substrate has a thickness of no more than approximately 180 micrometers, such that the apparatus has a total thickness of no more than approximately 25 millimeters.

44. The wearable ultrasound apparatus according to embodiments 1-43, wherein the at least one resilient substrate provides a plurality of signal traces embedded within or over said resilient substrate.

45. The wearable ultrasound apparatus according to embodiments 1-44, further comprising a near-infrared spectroscopy (“NIRS”) module positioned on the at least one resilient substrate, in electrical communication with the at least one controller, and configured for monitoring oxygenation status and/or biochemical measurements of the target site.

46. The wearable ultrasound apparatus according to embodiments 1-45, wherein the NIRS module comprises an at least one photodetector and an at least one near-infrared light emitting diode (“LED”).

47. The wearable ultrasound apparatus according to embodiments 1-46, wherein at least one of the at least one controller and ultrasound transceiver is positioned within an at least one microelectronics module.

48. The wearable ultrasound apparatus according to embodiments 1-47, further comprising a covering configured for protecting each of the ultrasound module, EP module, NIRS module, and at least one microelectronics module.

49. The wearable ultrasound apparatus according to embodiments 1-48, wherein the apparatus is configured as a self-contained wearable patch capable of being selectively engaged directly or indirectly with the target site.

50. A wearable ultrasound apparatus positionable on a target site of a user's body and comprising: an at least one ultrasound module configured for obtaining an at least one ultrasound image of the target site, the ultrasound module comprising: an at least one ultrasound transducer positioned on an at least one resilient substrate, the at least one ultrasound transducer comprising an at least one piezoelectric sensor, each of the at least one piezoelectric sensor comprising a piezoelectric material sandwiched between two or more electrodes; a pair of conductive layers positioned for substantially sandwiching the at least one piezoelectric sensor therebetween; and an at least one ultrasound transceiver in electrical communication with the at least one piezoelectric sensor; an at least one electrophysiological (“EP”) module positioned on the at least one resilient substrate and configured for detecting bioelectric signals of the target site; an at least one controller in electrical communication with each of the ultrasound module and EP module via the conductive layers, the at least one controller configured for selectively causing the ultrasound module to obtain at least one ultrasound image of the target site upon detection of bioelectric signal in the target site via the EP module.

51. A wearable ultrasound apparatus positionable on a target site of a user's body and comprising: an at least one ultrasound module configured for obtaining an at least one ultrasound image of the target site, the ultrasound module comprising: an at least one ultrasound transducer positioned on an at least one resilient substrate, the at least one ultrasound transducer comprising an at least one sensor; an at least one conductive layer positioned in electrical communication with the at least one sensor; and an at least one ultrasound transceiver in electrical communication with the at least one sensor; at least one of an electrophysiological (“EP”) module positioned on the at least one resilient substrate and configured for detecting bioelectric signals of the target site, and a near-infrared spectroscopy (“NIRS”) module positioned on the at least one resilient substrate and configured for monitoring oxygenation status and/or biochemical measurements of the target site; an at least one controller in electrical communication with each of the ultrasound module and at least one of the EP module and NIRS module via the conductive layers, the at least one controller configured for selectively causing the ultrasound module to obtain at least one ultrasound image of the target site upon detection of at least one of bioelectric signal, oxygenation status and/or biochemical measurement in the target site.

In closing, regarding the exemplary embodiments of the present invention as shown and described herein, it will be appreciated that a wearable ultrasound apparatus is disclosed and configured for use in connection with various biomedical applications, including musculoskeletal (“MSK”) imaging and analysis. Because the principles of the invention may be practiced in a number of configurations beyond those shown and described, it is to be understood that the invention is not in any way limited by the exemplary embodiments, but is generally directed to a wearable ultrasound apparatus and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that the logic code, programs, modules, processes, methods, and the order in which the respective elements of each method are performed are purely exemplary. Depending on the implementation, they may be performed in any order or in parallel, unless indicated otherwise in the present disclosure. Further, the logic code is not related, or limited to any particular programming language, and may comprise one or more modules that execute on one or more processors in a distributed, non-distributed, or multiprocessing environment. Additionally, the various illustrative logical blocks, modules, methods, and algorithm processes and sequences described in connection with the embodiments disclosed herein can be implemented as electronic hardware, computer software, or combinations of both. To clearly illustrate this interchangeability of hardware and software, various illustrative components, blocks, modules, and process actions have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of this document.

The phrase “non-transitory,” in addition to having its ordinary meaning, as used in this document means “enduring or long-lived.” The phrase “non-transitory computer readable medium,” in addition to having its ordinary meaning, includes any and all computer readable mediums, with the sole exception of a transitory, propagating signal. This includes, by way of example and not limitation, non-transitory computer-readable mediums such as register memory, processor cache and random-access memory (“RAM”).

The methods as described above may be used in the fabrication of integrated circuit chips. The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher level carrier) or in a multi-chip package (such as a ceramic carrier that has either or both surface interconnections or buried interconnections). In any case, the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A wearable ultrasound apparatus positionable on a target site of a user's body, the apparatus comprising:
an ultrasound module configured for obtaining an ultrasound image of the target site, the ultrasound module comprising:
a) an ultrasound transducer positioned on a resilient substrate, the ultrasound transducer comprising at least one sensor;
b) a conductive layer positioned in electrical communication with the at least one sensor;
c) an ultrasound transceiver in electrical communication with the sensor;
an electrophysiological (“EP”) module positioned on the at least one resilient substrate and configured for detecting bioelectric signals of the target site;
a controller in electrical communication with each of the ultrasound module and EP module via the conductive layers, the controller configured for selectively causing the ultrasound module to obtain at least one ultrasound image of the target site upon detection of bioelectric signal in the target site via the EP module, and
a near-infrared spectroscopy (“NIRS”) module positioned on the resilient substrate in electrical communication with the controller and configured for monitoring oxygenation status and/or biochemical measurements of the target site.

2. The wearable ultrasound apparatus of claim 1, wherein the sensor is at least one of a piezoelectric or a microelectromechanical (“MEM”) sensor.

3. The wearable ultrasound apparatus of claim 2, wherein the at least one sensor is a piezoelectric sensor comprising a piezoelectric material sandwiched between two or more electrodes.

4. The wearable ultrasound apparatus of claim 1, wherein the ultrasound module further comprises a matching layer positioned on a bottom surface of the at least one sensor and configured for providing acoustic impedance adaption.

5. The wearable ultrasound apparatus of claim 1, wherein the ultrasound module further comprises a backing layer positioned in contact with a top surface of the at least one sensor furthest from the target site, the at least one backing layer configured for absorbing ultrasound waves radiated by the at least one sensor that are not directed toward the target site.

6. The wearable ultrasound apparatus of claim 1, wherein the at least one ultrasound module further comprises an encapsulation layer positioned to isolate the conductive layer from the surrounding environment.

7. The wearable ultrasound apparatus of claim 1, further comprising a coupling layer positioned in contact with a bottom surface of a bottom most ultrasound transducer and configured for selectively adhering the apparatus to the target site.

8. The wearable ultrasound apparatus of claim 7, wherein the coupling layer has an acoustic impedance that approximates an acoustic impedance of the target site so as to provide impedance matching.

9. The wearable ultrasound apparatus of claim 1, wherein the ultrasound transducer comprises a plurality of adjacently arranged sensors configured as an array.

10. The wearable ultrasound apparatus of claim 9, wherein the ultrasound transducer comprises a plurality of arrays positioned in a side-by-side arrangement.

11. The wearable ultrasound apparatus of claim 9, wherein the ultrasound transducer comprises a plurality of arrays arranged so as to acquire orthogonal cross-sections of the target site.

12. The wearable ultrasound apparatus of claim 9, wherein the array is configured as a curve.

13. The wearable ultrasound apparatus of claim 9, wherein a subset of contiguous sensors are configured for being simultaneously activated on demand.

14. The wearable ultrasound apparatus of claim 9, wherein a plurality of sensors are sandwiched between a plurality of electrodes arranged in a row-column configuration so as to form a quasi-two-dimensional array.

15. The wearable ultrasound apparatus of claim 1, wherein the apparatus comprises a relatively greater quantity of sensors than ultrasound transceivers, such that the ultrasound transceiver is in electrical communication with a plurality of sensors.

16. The wearable ultrasound apparatus of claim 15, further comprising an at least one bidirectional multiplexer in electrical communication with the ultrasound transceiver and the corresponding plurality of sensors.

17. The wearable ultrasound apparatus of claim 1, wherein the EP module comprises at least one electrode array, in electrical communication with at least one signal conditioning circuit, and configured for detecting bioelectric signals of the target site.

18. A wearable ultrasound apparatus positionable on a target site of a user's body and comprising:
an ultrasound module configured for obtaining an ultrasound image of the target site, the ultrasound module comprising:
an ultrasound transducer positioned on a resilient substrate, the ultrasound transducer comprising at least one piezoelectric sensor, each of the at least one piezoelectric sensor comprising a piezoelectric material sandwiched between two or more electrodes;
a pair of conductive layers positioned for substantially sandwiching the at least one piezoelectric sensor therebetween; and
an ultrasound transceiver in electrical communication with the at least one piezoelectric sensor;
an electrophysiological (“EP”) module positioned on the resilient substrate and configured for detecting bioelectric signals of the target site; and
a controller in electrical communication with each of the ultrasound module and EP module via the conductive layers, the controller configured for selectively causing the ultrasound module to obtain at least one ultrasound image of the target site upon detection of a bioelectric signal in the target site via the EP module.

19. A wearable ultrasound apparatus positionable on a target site of a user's body and comprising:
an ultrasound module configured for obtaining an ultrasound image of the target site, the ultrasound module comprising:
an ultrasound transducer positioned on a resilient substrate, the ultrasound transducer comprising at least one sensor;
a conductive layer positioned in electrical communication with the at least one sensor; and
an ultrasound transceiver in electrical communication with the at least one sensor;
an electrophysiological (“EP”) module positioned on the resilient substrate and configured for detecting bioelectric signals of the target site, and a near-infrared spectroscopy (“NIRS”) module positioned on the resilient substrate and configured for monitoring oxygenation status and/or biochemical measurements of the target site; and
a controller in electrical communication with each of the ultrasound module and the EP module and NIRS module via the conductive layers, the controller configured for selectively causing the ultrasound module to obtain an ultrasound image of the target site upon detection of at least one of a bioelectric signal, an oxygenation status and/or a biochemical measurement in the target site.
